(19) United States
(12) Patent Application Publication
(10) Pub. No.: US 2026/0232948 A1
(43) Pub. Date: Aug. 13, 2026

(54) TRANSCUTANEOUS AURICULAR VAGAL NERVE STIMULATION FOR ENHANCED LEARNING

(71) Applicants: Washington University, St. Louis, MO (US); Saint Louis University, St. Louis, MO (US)

(72) Inventors: Eric Leuthardt, St. Louis, MO (US); Kara Donovan, St. Louis, MO (US); Jenna Gorlewicz, St. Louis, MO (US); Joshua Adams, St. Louis, MO (US)

(21) Appl. No.: 19/151,816

(22) PCT Filed: Feb. 5, 2024
(86) PCT No.: PCT/US2024/014452

§ 371 Date: Jul. 29, 2025

Related U.S. Application Data
(60) Provisional application No. 63/483,050, filed on Feb. 3, 2023.

(51) Int. Cl. A61M 21/00; G09B 5/02; G16H 20/30

(52) U.S. Cl. CPC A61M 21/00; G09B 5/02; G16H 20/30; A61M 2021/0022; A61M 2210/0662; A61M 2230/63

(57) ABSTRACT

A method of enhanced learning is provided. The method includes stimulating a cutaneous distribution of a subject's vagus nerve within the subject's ear with a nerve stimulating vibrational signal. The method also includes instructing the subject in performing an activity while the subject's vagus nerve is stimulated. The method further includes monitoring one or more statistics of the subject during the activity.

### CROSS REFERENCE TO RELATED APPLICATIONS

This application claims priority to U.S. Provisional Application No. 63/483,050, filed Feb. 3, 2023, which is hereby incorporated by reference in its entirety.

### BACKGROUND

The present disclosure generally relates to systems and methods of rehabilitating a motor function in a motor-impaired subject or enhancing motor function and performance in a normal subject, or enhancing memory and learning function in a cognitively-impaired or normal subject.

Each year, over 50% of the nearly 800,000 Americans that suffer a stroke will experience prolonged upper limb motor deficits six months post-injury. While an abundance of stroke rehabilitation strategies already exists, there remain at least several barriers that limit subjects' ability to improve. Many stroke rehabilitation strategies are dependent on the subject having considerable residual motor function, rendering them ineffective for those with severe hemiparesis or complete hemiplegia. Also, the vast majority of approaches do not use the subject's own neuronal activity, thus limiting their ability to maximize their central plasticity.

Vagus nerve stimulation (VNS) has emerged as a tool to promote and accelerate neuroplasticity in both healthy and injured brains, attributed in part to the release of plasticity-promoting neuromodulators at the cellular level. The vagus nerve is a mixed-fiber nerve that affects many upstream cortical and subcortical structures. Non-invasive transcutaneous auricular VNS (taVNS) has been demonstrated to improve post-stroke functional recovery. Despite encouraging preclinical and clinical results, the neural response to non-invasive VNS and the mechanism through which it affects functional motor recovery remain poorly understood in humans. This gap has limited the advancement of this therapeutic strategy. A well-characterized neural response to VNS will be critical in translating this approach in the chronic stroke population at scale. Vagus nerve stimulation has also been shown to contribute to enhanced cognitive function, such as memory performance, in human subjects, likely in part due to activations in key memory-related brain regions such as the hippocampus and amygdala.

Existing technology for transcutaneous auricular vagus nerve stimulation delivers surface electrical stimulation to regions of the ear that are innervated by the auricular branch of the vagus nerve. However, the electrical nature of the stimulation presents some drawbacks in that not all subjects may tolerate it and, in some cases, could find it uncomfortable or painful. Additionally, a critical factor in electrical stimulation is impedance—the contact quality between the electrode itself and the skin. Due to the heterogeneity of ear shapes and sizes in the population, impedance varies greatly from person to person and may have undesired effects on the quality or magnitude of the stimulation. Accordingly, a methodology for providing consistent stimulation of the vagus nerve is desired.

### BRIEF DESCRIPTION

In a first aspect, a method of enhanced learning is provided. The method includes stimulating a cutaneous distribution of a subject's vagus nerve within the subject's ear with a nerve stimulating vibrational signal. The method also includes instructing the subject in performing an activity while the subject's vagus nerve is stimulated. The method further includes monitoring one or more statistics of the subject during the activity.

In a second aspect, a system for enhanced learning is provided. The system includes a vibrotactile stimulation device including at least one form factor. The vibrotactile stimulation device is configured to provide a vibration to stimulate a subject's vagus nerve with a vibrotactile signal during an activity. The vibrotactile signal is configured to stimulate a cutaneous distribution of a subject's vagus nerve within the subject's ear with a nerve stimulating signal.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 illustrates a system for providing vagal nerve stimulation to a subject in accordance with at least one embodiment.

FIGS. 2A-2F illustrate exemplary form factors shown in FIG. 1 for non-invasive transcutaneous vagus nerve stimulation using the system shown in FIG. 1.

FIG. 3 illustrates a process for providing vagal nerve vibration stimulation using the system shown in FIG. 1.

FIG. 4 illustrates an example configuration of a client system shown in FIG. 1, in accordance with one embodiment of the present disclosure.

There are shown in the drawings arrangements that are presently discussed, it being understood, however, that the present embodiments are not limited to the precise arrangements and instrumentalities shown. While multiple embodiments are disclosed, still other embodiments of the present disclosure will become apparent to those skilled in the art from the following detailed description, which shows and describes illustrative aspects of the disclosure. As will be realized, the invention is capable of modifications in various aspects, all without departing from the spirit and scope of the present disclosure. Accordingly, the drawings and detailed description are to be regarded as illustrative in nature and not restrictive.

### DETAILED DESCRIPTION

In various aspects, devices and methods of for applying vagus nerve stimulation (VNS) to a subject. Due to the issues with the invasiveness of direct VNS, non-invasive vagus nerve stimulation is desired. In some aspects, the non-invasive vagus nerve stimulation device may be any known non-invasive vagus nerve stimulation device without limitation. This could include electrical stimulation, vibration, or ultrasonic methods of activating the nerve. However, for the purposes of this disclosure, the non-invasive vagus nerve stimulation device described herein provides vibrotactile stimulation to the subject. Non-limiting examples of non-invasive vagus nerve stimulation devices suitable for use in the systems and methods disclosed herein include a transcutaneous auricular vagus nerve stimulation (taVNS) device.

Due to issues with electrical non-invasive vagus nerve stimulation, the systems and methods described herein administer vibrotactile stimulation to the outer ear to stimulate the vagus nerve.

The devices described herein deliver vibrotactile stimulation to ear regions innervated by the auricular branch of the vagus nerve. In some embodiments, the device does not require user intervention once it is placed and does not have to be held in place during the stimulation. This advantage eliminates the potential for user error or inherent subjectivity in pressure (which may exist in an alternative means of delivering vibration to the ear). For example, a critical factor in electrical stimulation is impedance—the contact quality between the electrode itself and the skin. Due to the heterogeneity of ear shapes and sizes in the population, impedance varies greatly from person to person and may have undesired effects on the quality or magnitude of the stimulation.

To address these factors, we have developed a novel device for delivering vibrotactile stimulation to ear regions innervated by the auricular branch of the vagus nerve. The device does not require user intervention once it is placed and does not have to be held in place during the stimulation, which eliminates the potential for user error or inherent subjectivity in pressure. The vibrotactile device elicits brain changes consistent with activation in brain regions critically associated with learning, such as the hippocampus. Because of its ease of application and the finding of hippocampal activation, this stimulation device and method could be paired with stimuli that can enhance a learning effect. Examples could include combining vibrotactile stimulation with digital content that is educational in nature. For example, this could include, but is not limited: 1) digital flash cards to help learn new information, 2) educational material for people with learning disabilities (dedicated reading materials for dyslexia), and/or 3) digital representations of social scenes, phobic content, and human interactions to help patients with autism, phobias, and PTSD. The vibrotactile device could also be paired with movement interactive devices that could facilitate motor learning in the setting of athletic training and/or motor rehabilitation (stroke, spinal cord injury). In addition, the stimulation of the vibrotactile device could be used to provide sound therapy for tinnitus.

In some aspects, the transcutaneous stimulation of the auricular branch of the vagus nerve is implemented using a vibrotactile device that provides vibration to stimulate the vagus nerve through an ear of the subject. Without being limited to any particular theory, the external ear is an effective position for non-invasive stimulation of the vagus nerve, where the auricular branch travels in the pinna of the ear. In one aspect, the ear clips used for the VNS treatment are positioned along the concha of the ear. In another aspect the device can be wholly configured to be affixed to the ear which include the vibration device, power, electronics, and wearable form factor.

In some embodiments, the vibrotactile device may potentially be integrated into other ear-based wearables such as ear buds or earphones that can promote learning and more easy access to auditory content.

In some embodiments vibrotactile ear stimulation can be used in conjunction with playing video games, such as to improve game performance. This could include being integrated into head mounted systems associated with video games such as headset and earphones. Alternatively, this could be independent of these wearable head set systems. The ear simulation can enhance working memory, attention, reaction times, arousal, and motor learning, all of which relate to improved capability in video games. This could enhance a player's personal performance in a given video game or enhance his or her performance in video competitions. Video game performance can be monitored while vibrotactile stimulation is performed and parameters can be modified based on that performance. These modifications can be implemented by the gamer, the video game, or through a machine learning algorithm that automates the updates. These could include changing duration of stimulation, timing of stimulation based on game parameters or experiences, and vibrotactile frequency or intensity. The device can be associated with software that provides metrics on the relationship between video game performance and vibrotactile stimulation and also provides suggestions for usage. Examples of video games include multiplayer video games, flight simulators, first person shooting video games, racing video games; athletic or sport simulation games, and/or strategy games. Similarly, other computer mediated interactions could be enhanced or modified through person to person video presence or virtual reality.

A further embodiment is the use of vibrotactile stimulation to enhance preparation for standardized test taking. Vibrotactile stimulation can be used through learning modules that are either provided through physical materials such as books and notecards or through software that provide practice tests, notecards, tutoring, explanations, strategy, and the like. Ongoing performance can be monitored before, during, and after the stimulation to adjust parameters and to monitor test taking performance. This method also applies to general education where remote learning is used. Stimulation can be performed while students receive instruction and/or practice test taking.

In various aspects, the VNS administered by the non-invasive vagus nerve stimulation device is configured to enhance a behavioral motor performance of the subject. Without being limited to any particular theory, VNS is thought to modify the brain activity of a subject in a manner such that the subject's motor learning and/or motor performance are enhanced. By way of non-limiting example, the VNS may increase neural activity in brain regions critical to motor learning based on anatomical projections from the nucleus tractus solitarius (NTS), where vagal fibers terminate. Non-limiting examples of anatomical projections from the NTS associated with motor learning include the amygdala, hippocampus, and prefrontal cortex. In various aspects, the increased neural activity may be characterized as increased low-frequency coherence in brain areas receiving input from the NTS

In various aspects, the vagus nerve stimulation is delivered as characterized by a VNS parameter including a stimulation frequency and amplitude. The VNS parameters may be any suitable value without limitation. In some aspects, the stimulation frequency ranges from about 1 Hz to about 120 Hz. In some aspects, the stimulation frequency is selected from 6, Hz, 20 Hz, 30 Hz, 40 Hz, and 120 Hz.

The systems and methods described herein introduce a practical method for enhancing working memory: stimulating the vagus nerve with vibration. The findings indicate the application of vibrotactile taVNS is most beneficial to working memory performance when the cognitive load is close to working memory capacity. Mechanistically, vibrotactile taVNS rescues the arousal decline during continuous utilization of working memory. In addition, vibrotactile taVNS elevates arousal levels to the optimal level for working memory performance.

In various aspects, the systems disclosed above may be used in a method for rehabilitating a motor function in a motor-impaired subject in need. The method includes providing a brain-computer interface (BCI) and a non-invasive vagus nerve stimulation device similar to the devices described above. The method further includes administering a motor function rehabilitation treatment to the subject using the BCI as described above. The method further includes administering a vagus nerve stimulation (VNS) to the subject during at least a portion of the motor function rehabilitation treatment as described above, wherein the VNS is configured to enhance a behavioral motor performance of the subject.

The disclosed systems and methods combine non-invasive VNS with a BCI-driven rehabilitation technique to further enhance functional recovery in chronic stroke patients. In the examples herein, the optimal taVNS parameters to enhance motor learning were determined by recording invasive cortical physiology. Without being limited to any particular theory, taVNS is thought to contribute to elevated coherence that increases brain activity and widespread connectivity, contributing to enhanced motor learning. The disclosed systems and methods provide a mechanism-driven approach to designing, optimizing, and clinically translating taVNS rehabilitation techniques to motor recovery in the setting of chronic stroke. Guided by encouraging findings from an invasively recorded non-human primate and behavioral human data as described in the examples herein, the optimal taVNS parameters to increase neuronal coherence in invasively monitored human subjects will be determined and the neurophysiology of the motor learning effect associated with taVNS will be defined. The effects of different stimulation parameters to determine the effects of stimulation frequency, pulse-width, and current intensity on the subjects' brain activity will be systematically evaluated.

In various aspects, devices and methods of treatment for rehabilitating a motor function in a motor-impaired subject in need. The motor impairment in the subject may result from any known injury, affliction, or disorder associated with motor impairment without limitation. Non-limiting examples of an injury, affliction, or disorder that may be rehabilitated using the systems and methods disclosed herein include a stroke such as a unilateral stroke, a spinal cord injury, a neuromuscular disorder, a traumatic brain injury, a limb amputation, a peripheral nerve injury, and any other associated with motor impairment.

In various aspects, the BCI device may be any suitable device suitable for administering a motor function rehabilitation treatment to the motor-impaired subject without limitation. In some aspects, the BCI device may be an assistive BCI device configured to restore lost functions in the subject. Non-limiting examples of lost functions that may be restored using an assistive BCI device include communication in locked-in syndrome (e.g., as a result of amyotrophic lateral sclerosis), movements in paralysis, eating and drinking despite quadriplegia using robotic actuators and/or functional electrical stimulation systems. In other aspects, the BCI device may be a rehabilitative BCI device configured to foster neuroplasticity through manipulation or self-regulation of neurophysiological activity facilitating motor recovery using neurofeedback. In some aspects, the BCI device may translate electric, magnetic or metabolic brain activity into control signals of external devices that may replace, restore, enhance, supplement or improve the natural neural output, and thereby modify an ongoing interaction between the brain and its external or internal environment. Non-limiting examples of BCI devices suitable for use in the systems and methods disclosed herein are described in U.S. Pat. Nos. 9,730,816 and 10,596,014, as well as U.S. Patent Application Publication No. 2020/0188139, the contents of which are incorporated by reference herein in their entirety. In various aspects, the BCI device may be any suitable device suitable for administering a motor function rehabilitation treatment to the motor-impaired subject without limitation.

Beyond motor rehabilitation these techniques of taVNS can be used to enhance motor function in neurologically normal human subjects. Specifically, taVNS can be performed in while doing motor tasks that require substantial repetition to master. Examples of these include athletic activities, musical activities, medical activities such as surgery, work related activities, or any complex movement. Examples of athletic activities include, but are not limited to, basketball, tennis, baseball, golf, running, volleyball, badminton, swimming, boxing, table tennis, skiing, ice skating, roller skating, cricket, rugby, pool, darts, football, bowling, ice hockey, surfing, martial arts, horse racing, snowboarding, skateboarding, cycling, archery, fishing, gymnastics, figure skating, rock climbing, sumo, wrestling, fencing, water skiing, jet skiing, weight lifting, scuba diving, snorkeling, wind surfing, sky diving, hang gliding, and bungee jumping. Examples of musical activities include, but are not limited to, playing a musical instrument, dancing, theatrical activities, ballet, opera, and singing. Examples of medical activities would include, but is not limited to, performance of a surgical procedure, clinical procedure, diagnostic procedure, and physical exam. Examples of a work-related activity would include, but is not limited to, technical work task such as, but not limited to, electrical, plumbing or carpentry or other physical work, such as, but not limited to, construction, welding, roofing, and/or other physical labor. In other embodiments, the activity could also be a mental learning activity, such as memorization, such as through flash cards, learning mathematics, practicing public speaking, and/or other learning activities.

Furthermore, in some embodiments, vibrotactile taVNS may also be used for improving working memory through neuromodulation of the arousal pathways. For example, vibrotactile taVNS delivered to the concha significantly improves performance of 4-back tasks where maximal working memory is used. Arousal, which may be measured as skin conductance and pupil diameter, generally declines over the course of the tasks; however, vibrotactile taVNS significantly rescues this arousal decline, leading to arousal levels corresponding to optimal working memory levels. Moreover, during high-cognitive-load tasks, pupil diameter and skin conductance level are higher when vibrotactile taVNS is delivered to the concha.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

Optional” or “optionally” means that the subsequently described event or circumstance may or may not occur, and that the description includes instances where the event occurs and instances where it does not.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Any publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

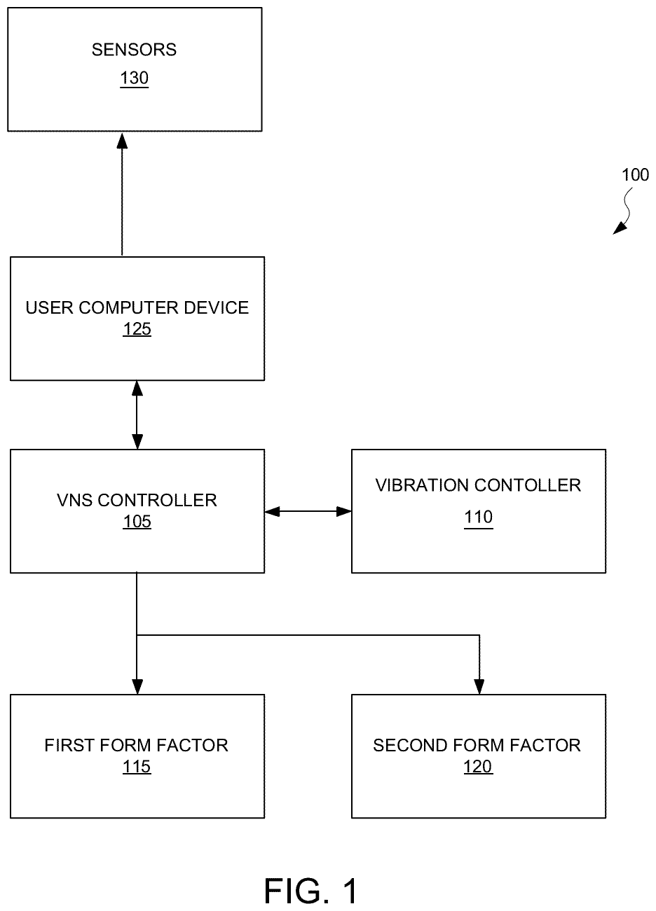

FIG. 1 illustrates a system 100 for providing vagal nerve stimulation to a subject in accordance with at least one embodiment.

A current trend in neuromodulation is the use of vagus nerve stimulation (VNS) as a means to promote neuroplasticity. The vagus nerve, which is comprised of 80% afferent fibers and 20% efferent fibers, is the main visceral sensory nerve and innervates many organs throughout the body. Stimulating the vagus nerve is typically performed using surgically implanted cuff electrodes—encircling the left vagus nerve within the carotid sheath—that are connected to a pulse generator implanted in the left side of the subject's chest. The left vagus nerve is used because it has fewer efferent fibers descending to the heart than the right vagus nerve, making it a safer site for stimulation. VNS has been proven effective as a treatment for intractable epilepsy and treatment-resistant depression, and has recently been investigated for several neurological injuries such as stroke and traumatic brain injury.

The vagus nerve is known to have a direct ascending projection to the nucleus tractus solitarius (NTS) which in turn activates the locus coeruleus (LC) and nucleus basalis (NB). The LC (located in the pons) and NB (located in the basal forebrain) are part of a neuromodulatory system with diffuse projections throughout cortical and subcortical areas. The LC contains noradrenergic neurons (norepinephrine, NE), and the NB contains cholinergic neurons (acetylcholine, ACh), both of which are known to be plasticity-promoting neuromodulators. The releases of NE and ACh are important in processes such as arousal, memory encoding, and task-related behavior, as well as processes requiring high attentional load. Thus, NE and ACh could have an important role in the mechanism of action for VNS-paired rehabilitation involving goal-directed behavior.

VNS stimulation triggers bursts of NE and ACh neuromodulator release causing changes in cortical plasticity. It is thought that these changes in cortical plasticity may lead to the therapeutic effect. Specifically, VNS has been shown to lead to reorganization of rat auditory and motor cortex, with increased cortical representations of VNS-paired tones or movements, respectively. This is further supported by lesion studies that have shown that depleting NE or ACh concentrations leads to blocked cortical plasticity and impaired learning. VNS has the capability to improve human recognition memory when administered at a moderate intensity. VNS has also been shown to improve retention on the Hopkins Verbal Learning Test when delivered during the memory consolidation phase, as well as to enhance working memory evidenced by reduced error rates on an executive functioning task.

While invasive VNS has been studied for several decades, non-invasive stimulation of the vagus nerve, specifically the auricular branch, which innervates the cymba concha and tragus regions of the outer ear, has emerged as an exciting non-invasive alternative. Transcutaneous auricular VNS (taVNS) provides clear benefits in eliminating the need for an invasive surgery and reducing the possible side effects which come with an implanted device. Several functional magnetic resonance imaging (fMRI) studies have demonstrated taVNS has central effects similar to invasive VNS. In comparison to sham earlobe stimulation, stimulating the left cymba concha has been shown to result in significant activation of the central vagal projections, such as the NTS and LC. Another fMRI study comparing the cymba concha and tragus as sites for taVNS found that both locations activated vagal projections, but only the cymba concha led to significant activations of the NTS and LC when compared to sham stimulation. Stimulating the vagus nerve via the outer ear has been investigated for many similar conditions as its invasive counterpart, such as epilepsy, depression, and tinnitus. Furthermore, similar to findings from invasive VNS, taVNS has also been shown to have cognitive benefits such as improved speech category learning and retention of non-native language tone categories, as well as enhanced associative memory in older adults.

The system 100 includes a VNS controller 105. The VNS controller 105 can be a computer device, such as a tablet, laptop, desktop, or other dedicated computer device including at least one processor in communication with at least one memory device. The VNS controller 105 can also include a user interface that that allows the VNS controller 105 to present information to a user and receive user inputs.

The VNS controller 105 is in communication with a vibration controller 110 configured to control the vibration for the vibrotactile stimulation. In some embodiments, the vibration controller 110 is in direct communication with the form factors 115 and 120. The VNS controller 105 can also be in communication with one or more vibrating form factors, such as a first form factor 115 and an optional second form factor 120. The first form factor 115 and the second form factor 120 are configured to provide the vibrotactile stimulation to the subject. In some embodiments, there is just a single form factor 115. In other embodiments, the first form factor 115 is applied to a first ear of the subject and the second form factor 120 is applied to the second ear of the subject. In the exemplary embodiments, the first form factor 115 and the second form factor 120 are secured to the subject to prevent accidental removal, but both are easy to apply and remove to the subject. In the exemplary embodiments, the first form factor 115 and the second form factor are temporarily attached to the subject's ear(s) to stimulate the vagus nerve.

In the exemplary embodiments, the form factors 115 and 120 provide stimulation to one or more of the cymba of the ear, the concha of the each, and the ear canal. In some embodiments, the form factors 115 and 120 are placed along the concha of the ear to stimulate the vagus nerve where the auricular branch travels in the pinna of the ear. In the exemplary embodiment, the form factor is attached to the subject's left ear.

In at least one embodiment, the VNS controller 105 is configured to provide treatment to the vagus nerve by vibrational stimulation for a period of twenty minutes. In at least one embodiment, one of the attributes of the vibrational stimulation is 6 Hz. The attributes of the vibrational stimulation stay the same throughout the treatment. In at least one further embodiment, the vibrational stimulation is performed twice a day. In at least one embodiment, the attributes of the vibrational stimulation are selected to maximize vagus somatosensory evoked potentials while avoiding perception of pain.

In the exemplary embodiment, the VNS controller 105 controls the output of the vibration controller 110 to provide the vibrational stimulation via the first form factor 115 and the second form factor 120.

In some further embodiments, VNS controller 105 is in communication with one or more user computer devices 125. The user computer device 125 may provide information to the VNS controller 105, such as one or more attributes of the subject that may alter the vibrational stimulation applied to the subject. Furthermore, the user computer device 125 may provide timing information to the VNS controller 105, such as when to apply the vibrational stimulation. Moreover, the user computer device 125 can receive information from the VNS controller 105, such as what were the attributes of the vibrational stimulation that was applied to the subject.

In some further embodiments, the first form factor 115 is applied to a first subject and the second form factor 120 is applied to a second subject. The VNS controller 105 controls the stimulation of each subject simultaneously. In some of these embodiments, the VNS controller 105 controls a plurality of form factors for a plurality of subjects receiving vibrotactile stimulation.

Sensors 130 can be used to determine the optimal taVNS parameters to enhance motor learning by recording invasive cortical physiology. The taVNS contributes to elevated gamma power that will increase brain activity and alertness, contributing to enhanced learning.

In some embodiments, the sensors 130 include stereotactic electroencephalography (sEEG). The sensors 130 can then be used to monitor the effects of stimulation parameters on the subject's brain activity, especially during motor or memory tasks. During these tasks, the sensors 130 may report the effect of stimulation frequency, etc. on the subject's brain activity. This may be used to find ideal parameters and/or adjust parameters to each individual subject. In at least one embodiment, parameter monitoring may be performed by monitoring a subject while they engage in a motor learning task paradigm (Serial Reaction Time Task, SRTT). In another embodiment, parameter monitoring may be performed by monitoring a subject while they engage in a memory-based task. The sensors 130 may also be used to monitor a subject's response and other attributes, such as, but not limited to, arousal state, behavioral response, skin conductance, and/or eye tracking. The user computer device 125 can collect electrophysiological, behavioral, and kinematic data in order to fully characterize the effects of taVNS on motor or memory learning. Other sensors 130 can include, but are not limited to, temperature, brain wave activity, galvanic response, blood pressure, heart rate, and/or any other attribute or statistic of the subject that is desired.

In at least one embodiment, the system 100 includes a form factor including an ear cup, a cushioning base that provides soft contact with the head, a T-head bolt that connects the contact tip and the ear cup via ear cup covers. In this embodiment, the vibration controller 110 controls an eccentric rotating mass (ERM) vibration motor of 5 mm diameter, which is held in at the contact tip. The motor's power cable passes through the T-head bolt. The T-head bolt allows individual adjustment of the depth of the contact. In addition, rotating the T-head bolt allows switching the stimulation target between the concha area and the earlobe. A digital stimulation box supplies 5V power, such as at 6 Hz to the eccentric Rotating Mass vibration motor. A study aiming to characterize locus coeruleus (LC) response to VNS has found that LC activity increases in response to stimulation frequencies ranging from 7.5 Hz to 120 Hz and that higher stimulation frequencies result in greater maximal discharge. As LC activity and performance of tasks have an inverted-U relationship, 6 Hz vibrotactile taVNS was chosen to increase Locus coeruleus activity and avoid exceeding the optimal value. An elastic strap stabilizes the vibrotactile device on the head. During the VNS session, vibrotactile stimulation is delivered to the concha area of the outer ear.

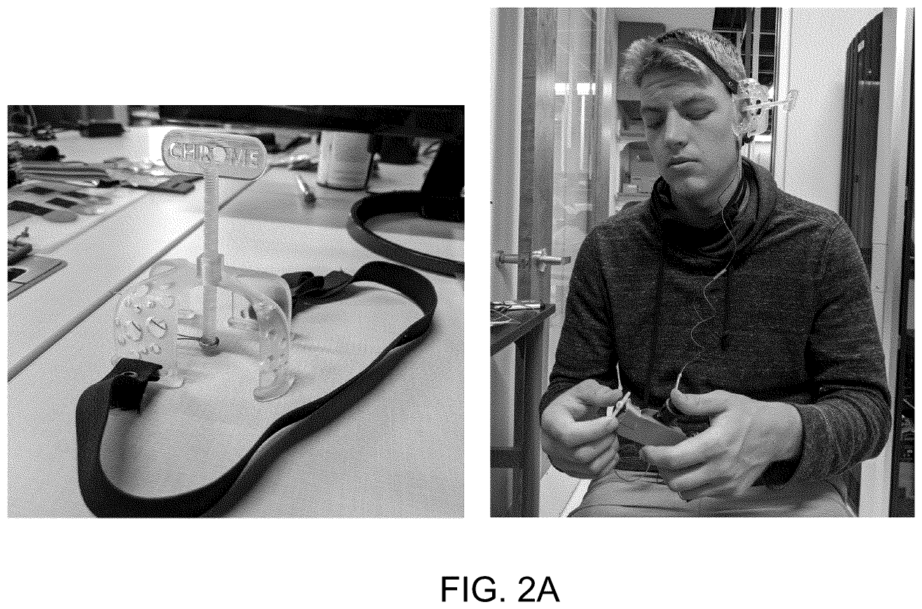

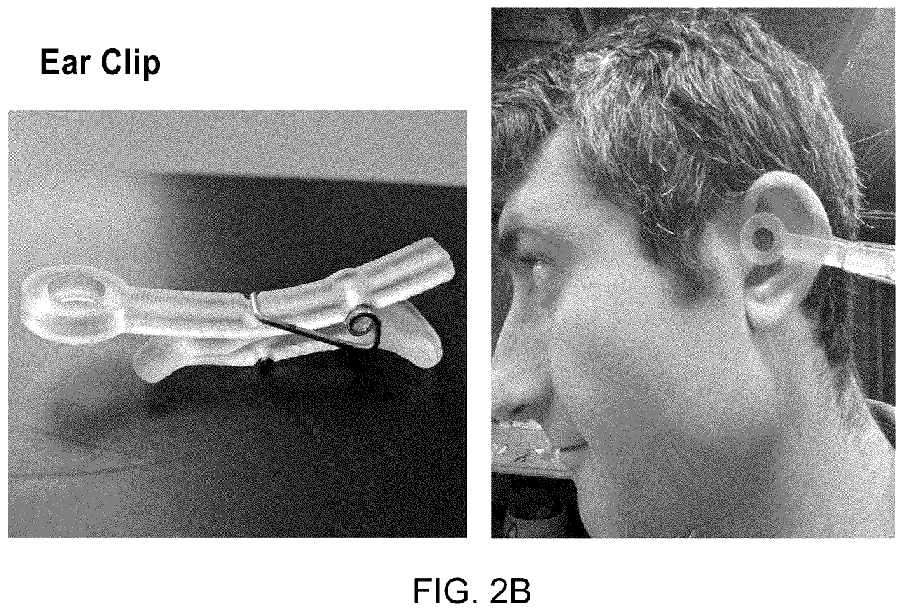

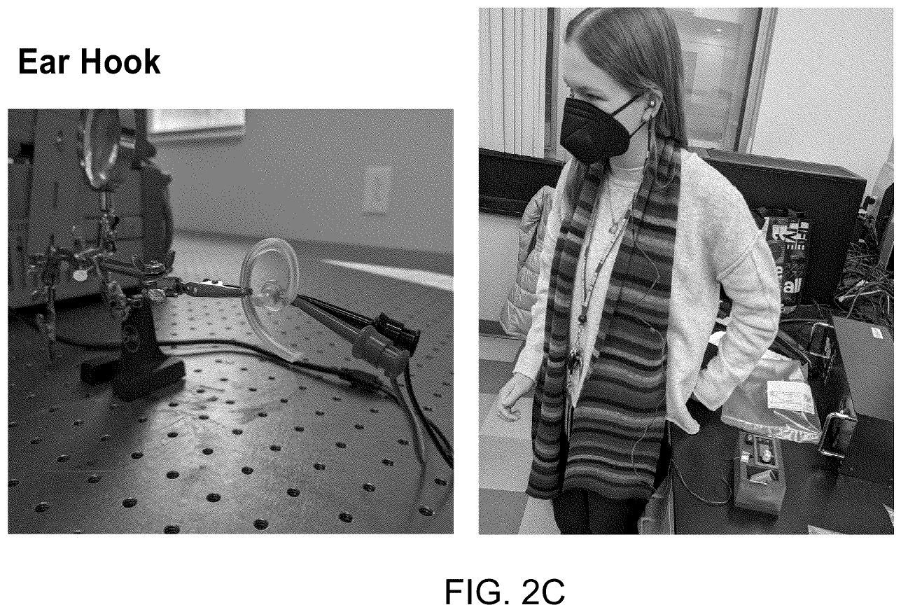

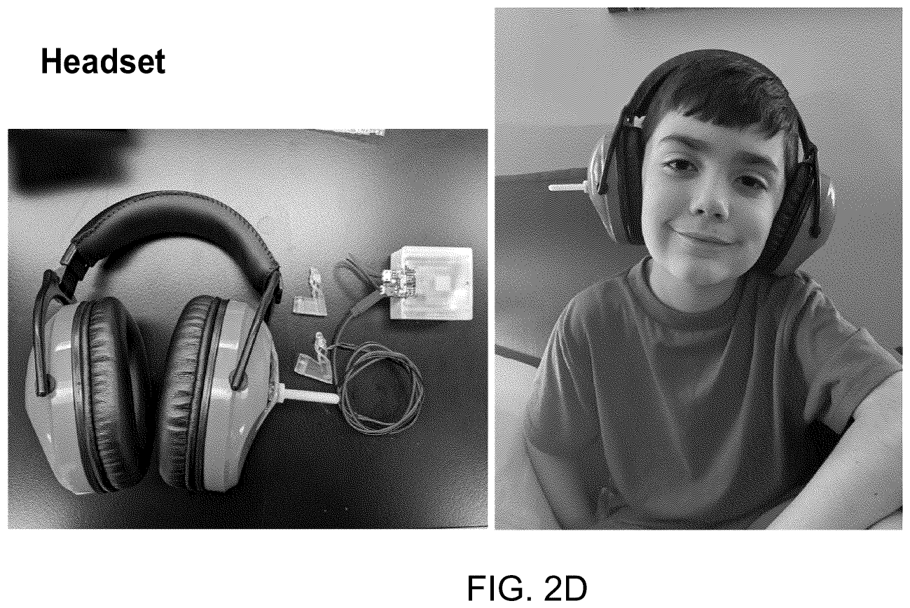

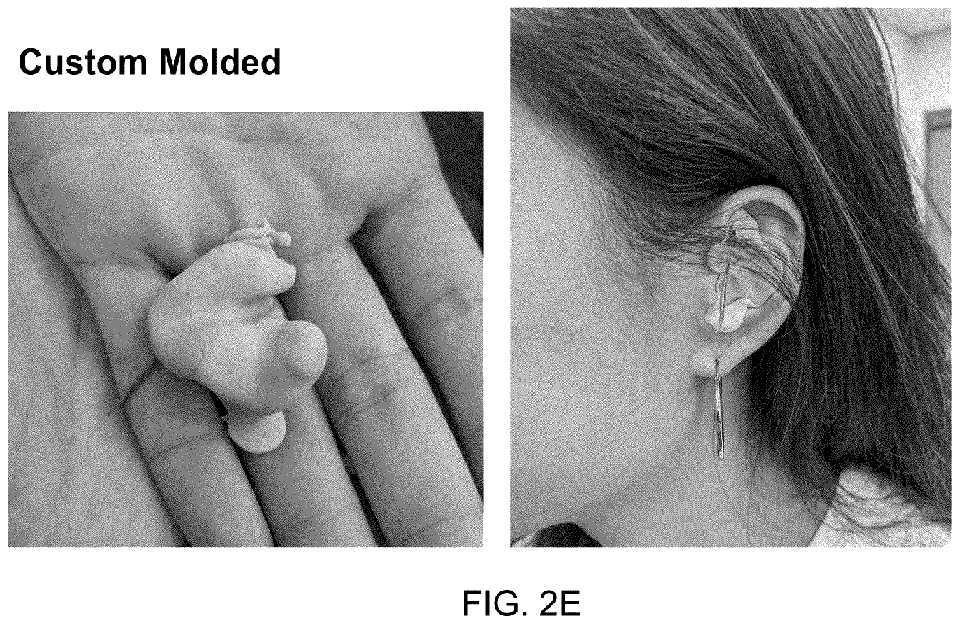

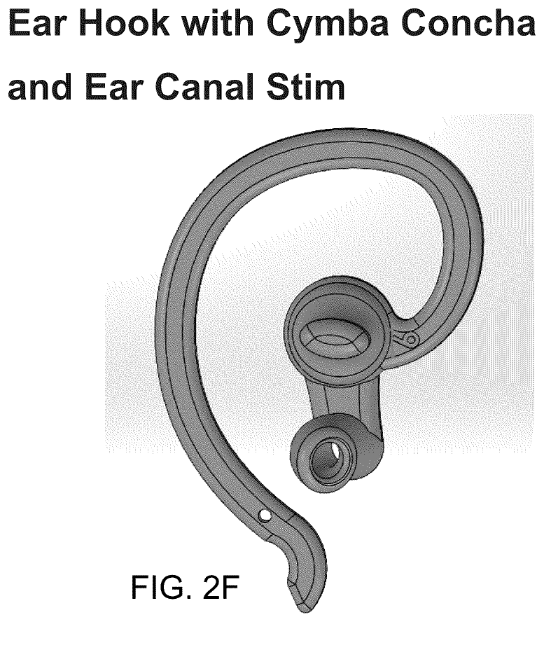

FIGS. 2A-2F illustrate exemplary form factors 115 (shown in FIG. 1) for non-invasive transcutaneous vagus nerve stimulation using the system 100 (shown in FIG. 1). In FIG. 2A, the form factor 115 is applied to the head with a headband to hold the form factor 115 in place. In FIG. 2B, the form factor 115 used is an ear clip to attach to the ear. In FIG. 2C, the form factor 115 used is an ear hook to attach to the ear. In FIG. 2D, the form factor 115 used is a headset with an integrated vibration generator to cover the ear. In FIG. 2E, the form factor 115 used is custom molded to be applied to a specific subject's ear. The custom molded form factor may include a modeling clay and/or wax that is moldable. In some of these embodiments, the vibration generator is encased in this moldable substance. In FIG. 2F, the form factor 115 illustrates an ear hook with stimulators for the cymba concha and the ear canal.

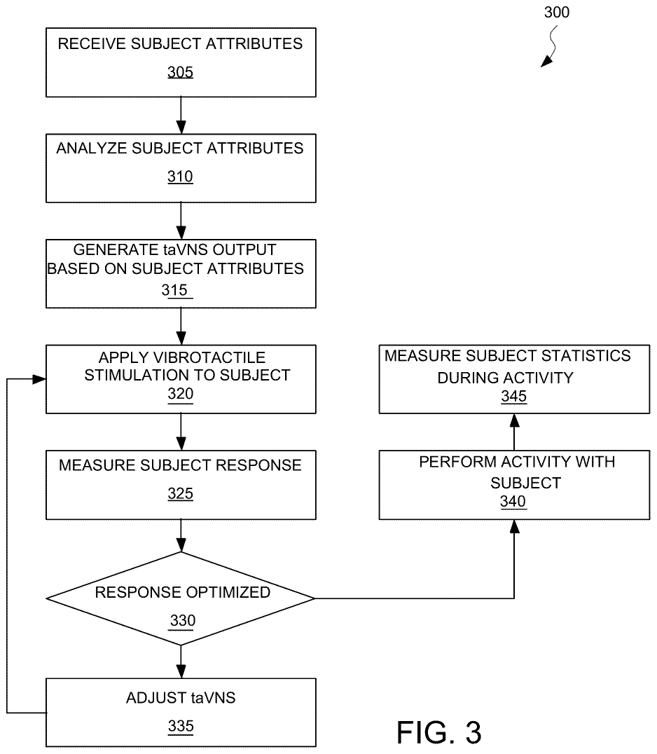

FIG. 3 illustrates a process 300 for providing vagal nerve vibration stimulation using the system 100 (shown in FIG. 1). In the exemplary embodiment, portions of process 300 are performed by a user computer device 125 (shown in FIG. 1), which may be, but is not limited to, a tablet, a laptop, a desktop, and/or and other computer device including at least one processor in communication with at least one memory device. Additionally, portions of process 300 are performed by the VNS controller 105 and/or the vibration controller 110 (both shown in FIG. 1).

In the exemplary embodiment, the user computer device 125 controls the vibration controller 110 to perform 320 vibration stimulation on a subject. During the VNS session, vibrotactile stimulation is delivered to the concha area of the outer ear. In at least one embodiment, the vibrotactile stimulation is applied at a starting value of 6 Hz.

In the exemplary embodiment, the user computer device 125 receives 305 subject attributes. The subject attributes could be received 305 when the subject arrives or by retrieving the subject's history. The subject attributes can include but are not limited to, height, weight, gender, heart rate, blood pressure, medical history, bloodwork results, vital statistics, presence/location of an aneurysm on vascular imaging, motor limitations, and other attributes. The subject attributes can further include CT (Computed tomography) imaging of stroke damaged nervous tissue. The subject attributes can be analyzed 310 to generate 315 the parameters for the taVNS vibrotactile stimulation based on the analyzed subject attributes.

In the exemplary embodiment, the user computer device 125 generates 315 appropriate parameters for taVNS vibrotactile stimulation of the subject based on historical analysis of a plurality of historical subjects and their response to different taVNS vibrotactile parameters and also based on their subject attributes. In some embodiments, the user computer device 125 trains an artificial intelligence and/or machine learning model based on the historical data for the plurality of subjects. In some of these embodiments, the model also includes historical information for the current subject based on previous sessions of vibrotactile stimulation.

The system 100 applies 320 vagal nerve vibrotactile stimulation to the subject. In some embodiments, the subject controls the session of vibrotactile stimulation to themself. In other embodiments, the session of vibrotactile stimulation is provided by another individual, such as, but not limited to, a healthcare provider. The form factor(s) 115 (shown in FIG. 1) are applied to the subject. In some embodiments, both form factors 115 and 120 are applied to the subject. In the exemplary embodiment, the first form factor 115 is applied to the concha of the left ear of the subject, as further shown in FIGS. 2A-2F. The VNS controller 105 then provides a vibration through the form factors 115 and 120. In the exemplary embodiment, the vibration is at 6 Hz. The frequency can range from 1 Hz to 200 Hz, or any other value as needed for the session of vibrotactile stimulation. Furthermore, other attributes of the frequency can change depending on other factors, such as the attributes of the subject. In some embodiments, the vibrotactile stimulation remains at the same attributes during the entire period of stimulation. In other embodiments, the vibrotactile stimulation is started at a lower frequency and the VNS controller 105 increases the frequency over time.

The sensors 130 measure 325 the subject response to the vibration stimulation. The subject response includes, but is not limited to detecting behavioral response, skin conductance, and eye tracking, and/or any other measurement desires for monitoring the subject response while the vibrotactile session in underway. The user computer device 125 determines 330 if the subject response is optimized, for example if subject arousal is optimized for performing 340 an activity. If the subject response is not optimized, the user computer device 125 adjusts 335 the vibrational stimulation, such as by increasing frequency of the vibration controller 110 until the sensors 130 detect that the subject response no longer changes with change in frequency or other conditions based on the desired usage of the session. In some embodiments, the sensors 130 can be also incorporated into the ear form factor so that physiologic signals can be acquired to regions adjacent to where ear vibration is being delivered.

When the subject response is optimized 330, the subject performs 340 an activity. In some embodiments, the subject performs a memory and/or learning based activity. In other embodiments, the subject performs a motor function activity, such as with a mechanical device to provide training to some of the subject's muscle and/or other desired response.

In at least one example, the activity takes up to twenty minutes. During that time, the VNS controller 105 applies 320 vibrotactile taVNS to the subject and measures 345 the subject's statistics during the activity. When the activity is over, the taVNS is discontinued, and the subject is disconnected from the form factors 115 and 120. In other embodiments, the activity or exercise treatment only takes a few minutes, but the length of the treatment increases incrementally over time, where the subject works up to being able to handle longer treatments. In other embodiments, the subject may receive treatment for one side of the body, the other, or both either serially or simultaneously. In some embodiments, the subject's statistics are monitored after the taVNS is discontinued to monitor how the subject's statistics return to baseline.

In one example, vibrotactile taVNS may be used for memory testing where subjects respond with a button press when they think a currently presented digit to be the same as the digit presented N trials prior. The digits presented in the N-back task are randomly selected from the pool of 4 digits: 0, 1, 2, and 3. Each subject completes three sessions of N-back tasks, including a baseline session, sham session, and VNS session. The order of sessions is randomly defined prior to subject recruitment. During the baseline session, the subject completes 8 blocks of N-back tasks with N ranging from 1 to 8. Each block includes 40 probes, that is, digit presentations when the subject needs to decide whether to press the button. Before the first probe, N digits were presented to load the participant's working memory. As digits were continuously presented on the screen, the subject needs to constantly update their working memory and make a decision whether to press the button. During the VNS or sham session, the subject completed 8 blocks of N-back tasks with N ranging from 0 to 4. The 0-back task in VNS or sham session is designed to be the control for attention. At the end of the baseline session, subjects were asked to rest and close their eyes. Data collected from subjects while they closed their eyes were used to ensure the validity of eye-tracking and skin conductance recording. During the VNS session, vibrotactile stimulation is delivered to the concha area of the outer ear. The ear lobe is mostly innervated by the great auricular nerve arising from the second and third cervical rami. Thus, in concordance with previous studies, the earlobe is used as the sham stimulation target.

In these sessions, behavioral responses were recorded using a push-button sensed by a multimodal trigger box, galvanic skin conductance using a USBAMP research amplifier, and pupillometry using an eye tracker.

In this example, hit rate, false alarm rate, reaction time, and d prime (d′) are used to evaluate performance. This refers to a probe as a digit presentation when the subject needs to decide whether to press the button. Hit rate refers to the proportion of actual positive probes when the participant presses the button, false alarm rate refers to the proportion of actual negative probes when the participant presses the button, and reaction time refers to the difference between the onset of digit presentation and the onset of button press. d′ is an integrative measure of hit rate and false alarm, quantifies the ability of a subject to discriminate between positive probes and negative probes, is calculated as follows:

$${d}^{′} = Z ( hit rate ) - Z ( false alarm rate ) \qquad EQ . 1$$

where Z is the inverse of the cumulative Gaussian distribution function.

In this example, the sensors 130 record skin conductance at a sampling rate of 1200 Hz and resampled it at 10 Hz for fast processing. The system 100 uses a 6th-order Butterworth low-pass filter with a cut-off frequency of 2 Hz to smooth the skin conductance and remove high-frequency artifacts. For event-related analysis, the skin conductance is scaled to a 0 to 1 range and aligned to the task onset for each N-back task block.

A convex optimization approach may be used to decompose the skin conductance signal into tonic and phasic components, which represent general arousal and sympathetic activities. To compare across subjects and sessions, skin conductance level is sampled every 10 seconds and z-scored based on the value in the 1-back task within each session. To bridge the behavioral effect and physiological effects of vibrotactile taVNS, the difference in skin conductance level between the sham/VNS session and baseline session is calculated for each difficulty. Next, performance metrics such as d′ are grouped based on the difference and compared the performance metrics.

The sensors 135 acquire pupil diameter at a rate of 120 Hz and measurement resolution of 0.1 mm. Invalid pupil measurements are removed that were induced due to blinks and closed eyes. The onset and offset of eye blinks were detected as those pupil diameter measurements that exceeded a threshold of 0.3 mm/ms. The blink is assumed to last no longer than 500 ms for better detection. If eye blink offset was not found within 500 ms following an onset, it was defined to occur 500 ms after the last detected eye blink onset. A safety margin for 15 ms is used around the eye blinks to account for potential pre-blink contamination of the pupillometry measurements. Finally, the remaining valid pupillometry measurement was resampled at 30 Hz for subsequent processing.

To elicit the temporal dynamic of pupil diameter under the VNS, sham, and baseline conditions, the inter-subject variance is eliminated by z-scoring the pupil diameter. The presentation of the digit and fixation cross is induced to a periodic component with a 1.5 s cycle into the subject's pupil diameter. To remove this confounding component and extract the general arousal component, 0.1 Hz low-pass filter is applied to the linearly interpolated pupil diameter. To show the effect of VNS on general arousal for N-back tasks of different difficulties, first the system interpolated and filtered the preprocessed pupil diameter using a 0.1 Hz low-pass filter. Subsequently, the system corrected the filtered pupil diameter by subtracting the mean of the filtered pupil diameter during the 1-back task for each session, which corrects the inter-session differences.

To identify the dynamic range of task performance, the system titrates the N-back task performance with N ranging from 1 to 8 during the baseline session. The hit rate and d′ decreased asymptotically as N increased. Reaction time only shows a clear increase when N changes from 1 to 2. It appears that performance decreases as more working memory was needed to complete the task. Therefore, as N increases, that is, as difficulty increases, d′ approaches a lower bound, representing d′ when the subject presses buttons at chance. The 4-parameter log-logistic regression model fits well d′ (mean residual=0, residual standard error=2.5). T-tests show that d′ at n=3 is significantly higher than estimated parameter c in the 4-parameter log-logistic regression model (t=2.71, p-value<0.01), while d′ at n=4 is not (t=0.94, p-value=0.18). These results show that subjects actively performed N-back tasks and that the maximum working memory span is most likely reached at 4-back, which align with studies of working memory capacity.

Decreased performance as N increased until 4 indicates that required working memory is close to the working memory capacity at the 4-back task. This may be compared to performance of 4-back tasks to reveal the effect of vibrotactile taVNS on cognitively demanding tasks. Paired Wilcoxon tests showed that vibrotactile VNS led to 96% improvement in d′ calculated as a percentage change of mean d′ compared with baseline session (Cohen's d=0.63, Bonferroni-corrected p-value<0.01, n=20). d′ during sham sessions is not significantly different than d′ during baseline sessions (Cohen's d=0.26, Bonferroni-corrected p-value>0.05, n=20). Although d′ during VNS sessions is higher than d′ during sham sessions, the difference is not significant (Cohen's d=0.48, Bonferroni-corrected p-value>0.05, n=20). increased d′ when N=4 does not come with increased reaction time.

Linear regression identifies the effects of vibrotactile taVNS as well as session, order, and difficulty on d′. The model with the lowest AIC is a linear mixed-effect model that incorporates difficulty, session, order, and the interaction between session and difficulty as fixed effects and a random intercept to account for individual differences. The median of scaled residuals is 0, and the interquartile range goes from −0.70 to 0.69. T-tests with Satterthwaite's method showed significant effects of difficulty, order, the interaction between taVNS and difficulty, and taVNS (see FIG. 3c). Specifically, d′ decreased as difficulty increased. Importantly, the decreases were less during the VNS session as indicated by a significantly positive β_(N:VNS). Besides, d′ was higher in a later session as indicated by the positive β_order (see Supplementary FIG. 3). A negative β_VNS is observed, which indicates that vibrotactile VNS might not be helpful for tasks where maximum working memory is not required. For reaction time, the model with the lowest AIC includes difficulty, session, order, and difficulty as fixed effects and a random intercept to account for individual differences. Accordingly, reaction time increases with difficulty and decreased with order.

Vibrotactile taVNS improves working memory, consistent with previous findings from invasive and transcutaneous electrical vagus nerve stimulation. To test if modulation of arousal contributes to the improvement, skin conductance was used as a proxy for task engagement and arousal. It was observed that skin conductance increased before the N-back task, which might represent mental preparation for cognitive demand. To study the effect of vibrotactile VNS on the temporal dynamic of skin conductance, skin conductance for each N-back task is rescaled from 0 to 1 and aligned at task onset. It was found that the reduction of skin conductance is less steep during the VNS session, indicating that vibrotactile VNS rescued the withdrawal of engagement due to prolonged cognitive demand. Time-wise comparison was performed to identify the time interval of interest. It was found that the difference in normalized skin conductance between VNS and sham session was significant at the middle and at the end of the N-back task. The effect size (Cohen's d) exceeds 0.2 15 seconds after the task onset, which indicates the effect size is not trivial. The normalized skin conductance during the rest period between N-back tasks for the three sessions is similar. Further, normalized skin conductance is compared between sessions for the three time intervals representing the early, middle, and late stages of the task, respectively. The normalized skin conductance during VNS is higher than baseline and sham sessions during the middle stage (Cohen's d=0.24 for the comparison between VNS and sham Cohen's d=0.25 for the comparison between VNS and baseline). During the late stage, the normalized skin conductance during VNS remains higher (Cohen's d=0.30 for the comparison between VNS and sham; Cohen's d=0.16 for the comparison between VNS and baseline).

Skin conductance is thought to comprise two components: the phasic component reflecting sympathetic nervous system activity, and the tonic (i.e., skin conductance level) related to general arousal. To study the effect of vibrotactile VNS on general arousal and task engagement in task with various difficulties, the skin conductance level sampled at each 10 seconds is z-scored to the 1-back task in each session to enable comparison across subjects and sessions. It was found that the normalized skin conductance level is higher in VNS sessions when the maximum working memory span is reached. Specifically, in the 3-back task, the normalized skin conductance level in the VNS session is significantly higher than that in the baseline session (permutation test, p=0.02, Cohen's d=0.22, Bonferroni-corrected p=0.08). Normalized skin conductance level is significantly higher in VNS in the 4-back task (permutation test, Bonferroni-corrected p=0.03 and Cohen's d=0.23 for the comparison between VNS and sham session; Bonferroni-corrected p=0.02 and Cohen's d=0.27 for the comparison between VNS and baseline session). The results show that vibrotactile VNS increases general arousal. To test if the general arousal contributes to the task performance, baseline-corrected performance metric d′ was grouped based on the skin conductance level in N-back tasks. For each N-back task of the same difficulty, the mean normalized skin conductance during baseline was subtracted from mean normalized skin conductance during VNS and sham session. The same procedure was conducted on d′. It was found that there was an optimal skin conductance level for working memory task performance. Specifically, baseline-corrected d′ is significantly higher when the baseline-corrected skin conductance level is located above the mean skin conductance level but below one standard deviation of the skin conductance level in the baseline. Cohen's d of baseline-corrected d′ between [−σ, 0] and [0, σ] baseline-corrected skin conductance level is −0.49, and Bonferroni-corrected p is 0.02. Besides, the effect size of baseline-corrected d′ between [0, σ] and [σ, 2σ] baseline-corrected skin conductance level is 0.91, and Bonferroni-corrected p is 0.02. The above results show that an optimal arousal level exists for better performance in working memory task. It is also observed that the baseline-corrected skin conductance level is within [0, σ], mean hit rate is highest, mean false alarm rate is lowest, while mean reaction time is lowest, compared to other skin conductance level.

Pupil dilation has been shown to reliably track VNS-evoked basal-forebrain cholinergic axon activity in animal studies. It is hypothesized that pupil diameter reduction over time would be rescued by vibrotactile taVNS. The preprocessed pupil diameter shows two components, including a 1.5 Hz oscillatory component representing the effect of the size and luminance of the stimuli on the screen or the transient effect of vibrotactile taVNS, and a general trend representing the general arousal. It was observed that left and right pupils changed similarly. The mean absolute deviation is 0.19 with 0.11 standard deviation and the mean Pearson correlation coefficient is 0.93 with a 0.04 standard deviation. Therefore, the right pupil diameter was chosen as a measure of arousal.

To compare the temporal dynamic between sessions, we z-scored the pupil diameter. In addition to the periodic component due to digit presentation, it is observed that the general trend in VNS sessions is higher than in other sessions. A 0.1 Hz low-pass filter was used to reveal the general arousal change. It was found that the arousal level rose to a higher level after the task began and decreased less steeply during the VNS session. The tendency is similar to the temporal dynamic of skin conductance. Timewise comparison between VNS and sham session with t-tests shows that the pupil diameter is higher in VNS after the task began. The difference reached the peak 10 s after the task onset and at 30 s after the task onset. The temporal dynamic of pupil diameter was further broken down into three time intervals. Permutation tests show that normalized pupil diameter during VNS is higher than other sessions in the [1 s, 10 s] interval (between VNS and Baseline session: p-value=0.01, Cohen's d=0.35; between VNS and Sham: p-value=0.04, Cohen's d=0.29, see FIG. 5d). To reveal the effect of vibrotactile taVNS during tasks with different difficulties, the preprocessed pupil diameter was firstly interpolated and filtered with a 0.1 low-pass filter. To study how vibrotactile taVNS affects the relationship between pupil diameter and task difficulty, the mean of the filtered pupil diameter during the 1-back task was subtracted from filtered pupil diameter for each session. Consistent with the findings in skin conductance, the corrected pupil diameter was higher in the VNS session in the 4-back task (permutation test, Bonferroni-corrected p<0.01 and Cohen's d=0.25 for the comparison between VNS and sham session; Bonferroni-corrected p<0.01 and Cohen's d=0.32 for the comparison between VNS and baseline session).

In additional embodiments, the user computer device 125 receives a plurality of monitored statistics of the subject from previous treatments. The user computer device 125 analyzes the plurality of monitored statistics. The user computer device 125 determines one or more parameters of the nerve stimulating vibrotactile signal based on the analyzed monitored statistics

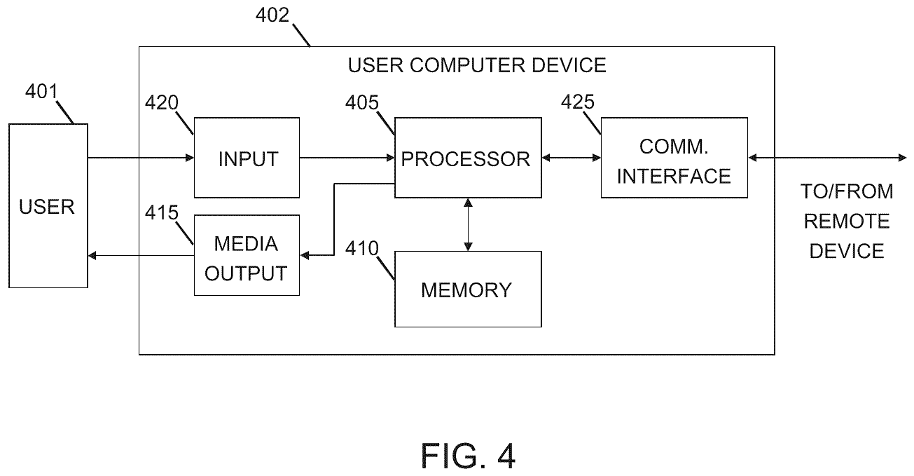

FIG. 4 illustrates an example configuration of a client system shown in FIG. 1, in accordance with one embodiment of the present disclosure. User computer device 402 is operated by a user 401. User computer device 402 may include, but is not limited to, VNS controller 105, vibration controller 110, and user computer device 125 (all shown in FIG. 1). User computer device 402 includes a processor 405 for executing instructions. In some embodiments, executable instructions are stored in a memory area 410. Processor 405 may include one or more processing units (e.g., in a multi-core configuration). Memory area 410 is any device allowing information such as executable instructions and/or transaction data to be stored and retrieved. Memory area 410 may include one or more computer-readable media.

User computer device 402 also includes at least one media output component 415 for presenting information to user 401. Media output component 415 is any component capable of conveying information to user 401. In some embodiments, media output component 415 includes an output adapter (not shown) such as a video adapter and/or an audio adapter. An output adapter is operatively coupled to processor 405 and operatively coupleable to an output device such as a display device (e.g., a cathode ray tube (CRT), liquid crystal display (LCD), light emitting diode (LED) display, or “electronic ink” display) or an audio output device (e.g., a speaker or headphones). In some embodiments, media output component 415 is configured to present a graphical user interface (e.g., a web browser and/or a client application) to user 401. A graphical user interface may include, for example, subject attributes or the attributes of the vibrational stimulation. In some embodiments, user computer device 402 includes an input device 420 for receiving input from user 401. User 401 may use input device 420 to, without limitation, select to apply the vibrational stimulation to the subject. Input device 420 may include, for example, a keyboard, a pointing device, a mouse, a stylus, a touch sensitive panel (e.g., a touch pad or a touch screen), a gyroscope, an accelerometer, a position detector, a biometric input device, and/or an audio input device. A single component such as a touch screen may function as both an output device of media output component 415 and input device 420.

User computer device 402 may also include a communication interface 425, communicatively coupled to a remote device such as a VNS controller 105 or a user computer device 125. Communication interface 425 may include, for example, a wired or wireless network adapter and/or a wireless data transceiver for use with a mobile telecommunications network.

Stored in memory area 410 are, for example, computer-readable instructions for providing a user interface to user 401 via media output component 415 and, optionally, receiving and processing input from input device 420. The user interface may include, among other possibilities, a web browser and/or a client application. Web browsers enable users, such as user 401, to display and interact with media and other information typically embedded on a web page or a website provided by a server. A client application allows user 401 to interact with, for example, VNS controller 105. For example, instructions may be stored by a cloud service and the output of the execution of the instructions sent to the media output component 415.

The methods and systems described herein may be implemented using computer programming or engineering techniques including computer software, firmware, hardware, or any combination or subset thereof, wherein the technical effects may be achieved by performing at least one of the following steps: a) stimulating the cutaneous distribution of a subject's vagus nerve within the ear with a nerve stimulating signal; b) instructing the subject in performing an activity while the subject's vagus nerve is stimulated; c) monitoring one or more statistics of the subject during the activity; d) the subject is performing a mental activity while the subject's vagus nerve is stimulated; e) assisting the subject in performing the physical activity; f) determining the movement performance for the subject; g) adjusting one or more parameters of the electrical signal to change the movement performance; h) the physical activity is assisted by a powered exoskeleton that assists and/or guides the movements of the subject; i) the activity is assisted by a virtual reality headset; j) the activity is assisted by a software visualization on a mobile computer device; k) the subject previously suffered a stroke; l) the subject previously suffered a spinal cord injury; m) the subject previously suffered a traumatic brain injury; n) the subject previously suffers from multiple sclerosis; n) the subject is neurologically normal and wants to enhance performance of a specific mental task; o) the specific mental task is a learning task; p) displaying flash cards to the subject as a part of the activity; q) subject is neurologically normal and wants to enhance motor performance of a specific task; r) the specific task is an athletic activity; s) the specific task is a musical activity; t) the specific task is a surgical activity; u) the specific task includes complex motor movement; v) the stimulation is vibrotactile stimulation of the vagus nerve; w) the stimulation is provided via a first form factor attached to the concha of the subject's ear; x) the first form factor is attached to the subject's left ear; y) the stimulation is provided to the auricular branch of the vagus nerve where the vagus nerve travels in the pinna of the ear; z) receiving a plurality of subject attributes associated with the subject; aa) analyzing the plurality of subject attributes; bb) determining one or more parameters of the electrical signal based on the analyzed subject attributes; cc) receiving a plurality of monitored statistics of the subject from previous treatments; dd) analyzing the plurality of monitored statistics; and ee) determining one or more parameters of the electrical signal based on the analyzed monitored statistics.

A computer program of one embodiment is embodied on a computer-readable medium. In an example, the system is executed on a single computer system, without requiring a connection to a server computer. In a further example embodiment, the system is being run in a Windows® environment (Windows is a registered trademark of Microsoft Corporation, Redmond, Washington). In yet another embodiment, the system is run on a mainframe environment and a UNIX® server environment (UNIX is a registered trademark of X/Open Company Limited located in Reading, Berkshire, United Kingdom). In a further embodiment, the system is run on an iOS® environment (iOS is a registered trademark of Cisco Systems, Inc. located in San Jose, CA). In yet a further embodiment, the system is run on a Mac OS® environment (Mac OS is a registered trademark of Apple Inc. located in Cupertino, CA). In still yet a further embodiment, the system is run on Android® OS (Android is a registered trademark of Google, Inc. of Mountain View, CA). In another embodiment, the system is run on Linux® OS (Linux is a registered trademark of Linus Torvalds of Boston, MA). The application is flexible and designed to run in various different environments without compromising any major functionality. In some embodiments, the system includes multiple components distributed among a plurality of computing devices. One or more components are in the form of computer-executable instructions embodied in a computer-readable medium. The systems and processes are not limited to the specific embodiments described herein. In addition, components of each system and each process can be practiced independently and separately from other components and processes described herein. Each component and process can also be used in combination with other assembly packages and processes.

As used herein, the terms “processor” and “computer” and related terms, e.g., “processing device”, “computing device”, and “controller” are not limited to just those integrated circuits referred to in the art as a computer, but broadly refers to a microcontroller, a microcomputer, a programmable logic controller (PLC), an application specific integrated circuit (ASIC), and other programmable circuits, and these terms are used interchangeably herein. In the embodiments described herein, memory may include, but is not limited to, a computer-readable medium, such as a random-access memory (RAM), and a computer-readable non-volatile medium, such as flash memory. Alternatively, a floppy disk, a compact disc-read only memory (CD-ROM), a magneto-optical disk (MOD), and/or a digital versatile disc (DVD) may also be used. Also, in the embodiments described herein, additional input channels may be, but are not limited to, computer peripherals associated with an operator interface such as a mouse and a keyboard. Alternatively, other computer peripherals may also be used that may include, for example, but not be limited to, a scanner. Furthermore, in the exemplary embodiment, additional output channels may include, but not be limited to, an operator interface monitor.

Further, as used herein, the terms “software” and “firmware” are interchangeable and include any computer program storage in memory for execution by personal computers, workstations, clients, servers, and respective processing elements thereof.

As used herein, the term “non-transitory computer-readable media” is intended to be representative of any tangible computer-based device implemented in any method or technology for short-term and long-term storage of information, such as, computer-readable instructions, data structures, program modules and sub-modules, or other data in any device. Therefore, the methods described herein may be encoded as executable instructions embodied in a tangible, non-transitory, computer readable medium, including, without limitation, a storage device, and a memory device. Such instructions, when executed by a processor, cause the processor to perform at least a portion of the methods described herein. Moreover, as used herein, the term “non-transitory computer-readable media” includes all tangible, computer-readable media, including, without limitation, non-transitory computer storage devices, including, without limitation, volatile and nonvolatile media, and removable and non-removable media such as a firmware, physical and virtual storage, CD-ROMs, DVDs, and any other digital source such as a network or the Internet, as well as yet to be developed digital means, with the sole exception being a transitory, propagating signal.

Furthermore, as used herein, the term “real-time” refers to at least one of the time of occurrence of the associated events, the time of measurement and collection of predetermined data, the time for a computing device (e.g., a processor) to process the data, and the time of a system response to the events and the environment. In the embodiments described herein, these activities and events may be considered to occur substantially instantaneously.

The aspects described herein may be implemented as part of one or more computer components, such as a client device, system, and/or components thereof, for example. Furthermore, one or more of the aspects described herein may be implemented as part of a computer network architecture and/or a cognitive computing architecture that facilitates communications between various other devices and/or components. Thus, the aspects described herein address and solve issues of a technical nature that are necessarily rooted in computer technology.

A processor or a processing element may be trained using supervised or unsupervised machine learning, and the machine learning program may employ a neural network, which may be a convolutional neural network, a deep learning neural network, a reinforced or reinforcement learning module or program, or a combined learning module or program that learns in two or more fields or areas of interest. Machine learning may involve identifying and recognizing patterns in existing data in order to facilitate making predictions for subsequent data. Models may be created based upon example inputs in order to make valid and reliable predictions for novel inputs.

Additionally or alternatively, the machine learning programs may be trained by inputting sample data sets or certain data into the programs, such as images, object statistics and information, traffic timing, previous trips, and/or actual timing. The machine learning programs may utilize deep learning algorithms that may be primarily focused on pattern recognition, and may be trained after processing multiple examples. The machine learning programs may include Bayesian Program Learning (BPL), voice recognition and synthesis, image or object recognition, signal processing, optical character recognition, and/or natural language processing—either individually or in combination. The machine learning programs may also include natural language processing, semantic analysis, automatic reasoning, and/or machine learning.

Supervised and unsupervised machine learning techniques may be used. In supervised machine learning, a processing element may be provided with example inputs and their associated outputs, and may seek to discover a general rule that maps inputs to outputs, so that when subsequent novel inputs are provided the processing element may, based upon the discovered rule, accurately predict the correct output. In unsupervised machine learning, the processing element may be required to find its own structure in unlabeled example inputs. In one embodiment, machine learning techniques may be used to determine brain responses to stimuli such as VNS settings.

Based upon these analyses, the processing element may learn how to identify characteristics and patterns that may then be applied to analyzing image data, model data, and/or other data. For example, the processing element may learn, to identify brain responses to stimuli and the VNS settings for different subjects to provide optimal gamma activity. The processing element may also learn how to identify trends that may not be readily apparent based upon collected traffic data, such as trends that identify when gamma activity will spike or decline.

The exemplary systems and methods described and illustrated herein therefore provide VNS treatments for improving the effectiveness of learning treatments.

The computer-implemented methods and processes described herein may include additional, fewer, or alternate actions, including those discussed elsewhere herein. The present systems and methods may be implemented using one or more local or remote processors, transceivers, and/or sensors (such as processors, transceivers, and/or sensors mounted on vehicles, stations, nodes, or mobile devices, or associated with smart infrastructures and/or remote servers), and/or through implementation of computer-executable instructions stored on non-transitory computer-readable media or medium. Unless described herein to the contrary, the various steps of the several processes may be performed in a different order, or simultaneously in some instances.

Additionally, the computer systems discussed herein may include additional, fewer, or alternative elements and respective functionalities, including those discussed elsewhere herein, which themselves may include or be implemented according to computer-executable instructions stored on non-transitory computer-readable media or medium.

In the exemplary embodiment, a processing element may be instructed to execute one or more of the processes and subprocesses described above by providing the processing element with computer-executable instructions to perform such steps/sub-steps, and store collected data (e.g., trust stores, authentication information, etc.) in a memory or storage associated therewith. This stored information may be used by the respective processing elements to make the determinations necessary to perform other relevant processing steps, as described above.

The aspects described herein may be implemented as part of one or more computer components, such as a client device, system, and/or components thereof, for example. Furthermore, one or more of the aspects described herein may be implemented as part of a computer network architecture and/or a cognitive computing architecture that facilitates communications between various other devices and/or components. Thus, the aspects described herein address and solve issues of a technical nature that are necessarily rooted in computer technology.

Although specific features of various embodiments may be shown in some drawings and not in others, this is for convenience only. In accordance with the principles of the systems and methods described herein, any feature of a drawing may be referenced or claimed in combination with any feature of any other drawing.

Some embodiments involve the use of one or more electronic or computing devices. Such devices typically include a processor, processing device, or controller, such as a general purpose central processing unit (CPU), a graphics processing unit (GPU), a microcontroller, a reduced instruction set computer (RISC) processor, an application specific integrated circuit (ASIC), a programmable logic circuit (PLC), a programmable logic unit (PLU), a field programmable gate array (FPGA), a digital signal processing (DSP) device, and/or any other circuit or processing device capable of executing the functions described herein. The methods described herein may be encoded as executable instructions embodied in a computer readable medium, including, without limitation, a storage device and/or a memory device. Such instructions, when executed by a processing device, cause the processing device to perform at least a portion of the methods described herein. The above examples are exemplary only, and thus are not intended to limit in any way the definition and/or meaning of the term processor and processing device.

The computer-implemented methods discussed herein may include additional, less, or alternate actions, including those discussed elsewhere herein. The methods may be implemented via one or more local or remote processors, transceivers, servers, and/or sensors, and/or via computer-executable instructions stored on non-transitory computer-readable media or medium.

Additionally, the computer systems discussed herein may include additional, less, or alternate functionality, including that discussed elsewhere herein. The computer systems discussed herein may include or be implemented via computer-executable instructions stored on non-transitory computer-readable media or medium.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. A method of enhanced learning, the method comprising:
stimulating a cutaneous distribution of a subject's vagus nerve within the subject's ear with a nerve stimulating vibrational signal;
instructing the subject in performing an activity while the subject's vagus nerve is stimulated; and
registering that the activity and the stimulation have been performed concurrently.

2. The method of claim 1, wherein the stimulation is vibrotactile stimulation of the vagus nerve at a frequency of 6 Hz.

3. The method of claim 1, wherein the stimulation is provided via a first form factor attached to the concha of the subject's ear.

4. The method of claim 3, wherein the first form factor is attached to the subject's left ear.

5. The method of claim 3, wherein the stimulation is provided to the auricular branch of the vagus nerve where the vagus nerve travels in the pinna of the ear.

6. The method of claim 1, wherein the subject is performing a mental activity while the subject's vagus nerve is stimulated.

7. The method of claim 1, wherein the subject in performing a physical activity while the subject's vagus nerve is stimulated, and wherein the method further comprises assisting the subject in performing a memory-based activity.

8. A method of enhanced learning, the method comprising:
stimulating a cutaneous distribution of a subject's vagus nerve within the subject's ear with a nerve stimulating vibrational signal;
instructing the subject to perform a physical activity while the subject's vagus nerve is stimulated; and
registering that the activity and the stimulation have been performed concurrently; and
assisting the subject in performing the physical activity.

9. The method of claim 8 further comprising:
determining movement performance for the subject; and
adjusting one or more parameters of the nerve stimulating signal to change the movement performance.

10. The method of claim 1, wherein the activity is assisted by a virtual reality headset.

11. The method of claim 1, wherein the activity is assisted by a software visualization on a mobile computer device.

12. The method of claim 1, wherein the subject previously suffered at least one of a stroke, a spinal cord injury, a traumatic brain injury, and multiple sclerosis.

13-15. (canceled)

16. The method of claim 1, wherein the subject is neurologically normal and wants to enhance performance of a specific mental task.

17. The method of claim 16, wherein the specific mental task is a learning task.

18. The method of claim 16 further comprising displaying flash cards to the subject as a part of the activity.

19. The method of claim 1, wherein the subject is neurologically normal and wants to enhance motor performance of a specific task.

20. The method of claim 19, wherein the specific task is at least one of an athletic activity, a musical activity, a surgical activity and a complex motor movement.

21-23. (canceled)

24. The method of claim 1 further comprising:
receiving a plurality of subject attributes associated with the subject;
analyzing the plurality of subject attributes; and
determining one or more parameters of the nerve stimulating signal based on the analyzed subject attributes.

25. The method of claim 1 further comprising:
receiving a plurality of monitored statistics of the subject from previous treatments;
analyzing the plurality of monitored statistics; and
determining one or more parameters of the nerve stimulating signal based on the analyzed monitored statistics.

26. A system for enhanced learning, the system comprising a vibrotactile stimulation device including at least one form factor, wherein the vibrotactile stimulation device is configured to provide a vibration to stimulate a subject's vagus nerve with a vibrotactile signal during an activity, wherein the vibrotactile signal is configured to stimulate a cutaneous distribution of a subject's vagus nerve within the subject's ear with a nerve stimulating signal.
